(19) United States
(12) Patent Application Publication
(10) Pub. No.: US 2009/0252845 A1
(43) Pub. Date: Oct. 8, 2009

(54) COLLIDER CHAMBER APPARATUS AND METHOD OF USE

(76) Inventor: Kenneth J. SOUTHWICK, Marshfield, MA (US)

Correspondence Address: WILMERHALE/BOSTON, 60 STATE STREET, BOSTON, MA, 02109, US

(21) Appl. No.: 12/061,872

(22) Filed: Apr. 3, 2008

(51) Int. Cl. A23L 3/00; A61L 2/04; B01F 15/06; B01F 13/10

(52) U.S. Cl. 426/330; 422/38; 366/147; 366/296; 366/144

(57) ABSTRACT

The disclosed apparatus includes a stator and a rotor disposed for rotation within the stator. An inner wall of the stator defines one or more collider chambers. Rotation of the rotor causes movement of fluid disposed between the rotor and stator and establishes a rotational flow pattern within the collider chambers. The fluid movement induced by the rotor increases the temperature, density, and pressure of the fluid in the collider chamber. Aspects of the invention include increasing the metals and/or solids content of the fluid.

### CROSS-REFERENCES TO RELATED APPLICATIONS

This application is related to U.S. patent application Ser. No. 11/030,272, filed Jan. 6, 2005, U.S. patent application Ser. No. 09/590,049, filed Jun. 8, 2000, now U.S. Pat. No. 6,855,299, and U.S. patent application Ser. No. 09/354,413, filed Jul. 15, 1999, now U.S. Pat. No. 6,110,432, all entitled Collider Chamber Apparatus and Method of Use and all incorporated by reference herein.

### BACKGROUND OF THE INVENTION

1. Field of the Invention

The present invention relates to a collider chamber apparatus. More specifically, the present invention relates to an apparatus and method for increasing the number of molecular collisions that occur in a fluid, using artificially induced movement to increase the heat of a fluid, and changing characteristics of the fluid to increase the susceptibility of the fluid to heating.

2. Description of the Related Art

Many devices are known that use motion to manipulate fluids. For example, common household blenders use rotary motion of a stirring blade to mix or froth fluids. As another example, U.S. Pat. No. 3,285,702 discloses a device for mixing fluids to increase chemical reactions between multiple reactants. As yet another example, centrifuges are known for using rotary motion to separate solid particles suspended in a fluid from the fluid. All these devices induce some type of motion in a fluid to change some of the fluid's properties in a desired fashion.

It is also known that application of heat to a fluid will increase the speed of molecules in that fluid. However, it has heretofore been unknown to use motion to produce fundamental changes in the properties of a fluid.

It is therefore an object of the present invention to provide a collider chamber apparatus for increasing and controlling the number of molecular collisions occurring in a fluid.

It is yet another object of the invention to provide a collider chamber apparatus that induces movement in a fluid and thereby increases the temperature of the fluid.

It is still another object of the invention to provide a collider chamber apparatus that adds kinetic energy to a fluid and converts that kinetic energy into thermal energy for heating and processing fluids.

### BRIEF SUMMARY OF THE INVENTION

These and other objects are provided by a collider chamber apparatus. The apparatus includes a rotor and a stator, and the stator defines a plurality of collider chambers. Rotation of the rotor induces cyclonic fluid flow patterns in each of the collider chambers.

Under an aspect of the invention, a method of heating includes disposing a fluid comprising a metals content of more than about 100 mg/L between a stator and a rotor. The stator includes an inner wall, the inner wall defines a plurality of collider chambers, and the rotor includes an outer wall that is proximal to the stator inner wall. The method also includes rotating the rotor, relative to the stator, about an axis. Rotation of the rotor in a first direction relative to the stator causes the fluid in each of the collider chambers to rotate within the collider chamber in a second direction opposite to the first direction. Rotation of the rotor causes the temperature of the fluid in the collider chambers to increase.

Under other aspects of the invention, the metallic species being ionic and/or colloidal. The metallic species can be aluminum, copper, and/or iron.

Under another aspect of the invention, a method of heating includes disposing a fluid comprising a total suspended solids of more than 370 mg/L between a stator and a rotor. The stator includes an inner wall, the inner wall defines a plurality of collider chambers, and the rotor includes an outer wall that is proximal to the stator inner wall. The method further includes rotating the rotor, relative to the stator, about an axis. Rotation of the rotor in a first direction relative to the stator causes the fluid in each of the collider chambers to rotate within the collider chamber in a second direction opposite to the first direction Rotation of the rotor causes the temperature of the fluid in the collider chambers to increase.

Under further aspects of the invention, rotating the rotor, relative to the stator, causes the material of the at least one of the rotor and stator to enter the fluid. The invention can further comprises providing the fluid comprising the metals content and/or total suspended solids content.

Under yet another aspect of the invention, a method of heating includes disposing a fluid between a stator and a rotor. The stator includes an inner wall, the inner wall defines a plurality of collider chambers, and the rotor includes an outer wall that is proximal to the stator inner wall. The method also includes rotating the rotor, relative to the stator, about an axis above a predetermined rotational speed for a cumulative predetermined amount of time. The cumulative predetermined amount of time is at least about 24 hours. The method further includes, after rotating the rotor for the cumulative predetermined amount of time, rotating the rotor, relative to the stator, about the axis. Rotation of the rotor in a first direction relative to the stator causes the fluid in each of the collider chambers to rotate within the collider chamber in a second direction opposite to the first direction. Rotation of the rotor causes the temperature of the fluid in the collider chambers to increase. In some aspects, the predetermined rotational speed can be at least about 180° rotations per minute.

Under still further aspects of the invention, a method of heating includes increasing a pressure of the fluid above a predetermined pressure before delivering the fluid to the at least one of the collider chambers. The predetermined pressure can be about 14.7 pounds per square inch absolute. The predetermined pressure can also be about 44.7 pounds per square inch absolute.

Under another aspect of the invention, a method includes providing a stator having an inner wall; the inner wall defines a plurality of collider chambers. The method also includes providing a rotor disposed for rotation about an axis; an outer wall of the rotor is proximal to the inner wall of said stator. The method further includes introducing a putatively contaminated fluid into a space between the inner wall of the stator and said outer wall of the rotor. The contaminated fluid includes an infectious agent selected from the group consisting of bacteria, virus, parasite, and a combination thereof. The method also includes rotating the rotor within the stator to generate a rotational flow of the fluid in each of the collider chambers. The rotational flow of the fluid in each of the collider chambers causes the temperature of at least portion of the fluid contained within each collider chamber to increase.

Still other objects and advantages of the present invention will become readily apparent to those skilled in the art from the following detailed description wherein several embodiments are shown and described. As will be realized, the invention is capable of other and different embodiments, and its several details are capable of modifications in various respects, all without departing from the invention. Accordingly, the drawings and description are to be regarded as illustrative in nature, and not in a restrictive or limiting sense, with the scope of the application being indicated by the claims appended hereto.

### BRIEF DESCRIPTION OF THE SEVERAL VIEW OF THE DRAWINGS

FIG. 1 shows a sectional side view of a collider chamber apparatus constructed according to the invention;

FIG. 1A shows a sectional side view of another embodiment of a collider chamber apparatus constructed according to the invention;

FIG. 2 shows a top sectional view of the collider chamber apparatus taken along line 2-2 of FIG. 1;

FIG. 3 shows a perspective view of the collider chamber apparatus shown in FIG. 1;

FIG. 4 shows a top view of a cyclonic flow pattern in a collider chamber constructed according to the invention;

FIG. 5 shows a perspective view of a cyclonic flow pattern in a collider chamber constructed according to the invention;

FIG. 6 shows a top view of another cyclonic flow pattern in a collider chamber constructed according to the invention;

FIG. 7 shows a top view of another cyclonic flow pattern in a collider chamber constructed according to the invention;

FIG. 8 shows a top view of alternative embodiment cyclonic flow pattern collider chambers constructed according to the invention;

FIG. 9 shows a top sectional view of a collider chamber apparatus constructed according to the invention in which each collider chamber is provided with its own fluid inlet, outlet, and control valves;

FIG. 10 shows a sectional side view of a collider chamber apparatus constructed according to the invention in which the rotor is characterized by an “hour-glass” shape;

FIG. 11 shows a sectional side view of another embodiment of a collider chamber apparatus constructed according to the invention;

FIG. 12 shows a sectional side view of another embodiment of a collider chamber apparatus constructed according to the invention; and

FIG. 13 shows a sectional view of the apparatus shown in FIG. 12 taken along line 13-13.

FIG. 14 shows a perspective view of a collider chamber apparatus with helical collider chambers.

FIG. 15 shows a semi-transparent perspective view of a collider chamber apparatus with a segmented stator.

FIG. 16 shows a semi-transparent exploded perspective view of the collider chamber apparatus of FIG. 15.

FIG. 17 shows a perspective view of one of the segments of the collider chamber apparatus of FIG. 15.

### DETAILED DESCRIPTION OF THE INVENTION

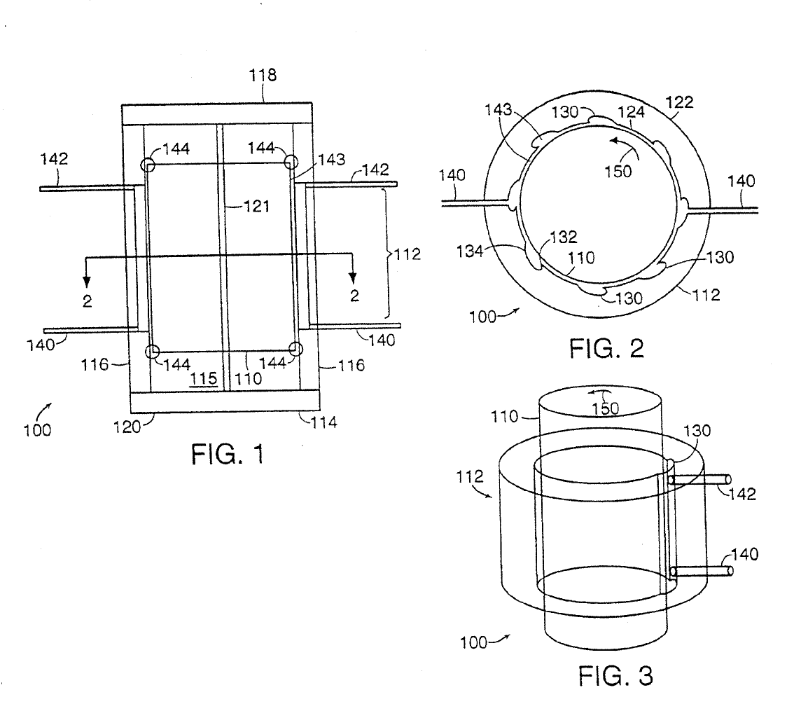

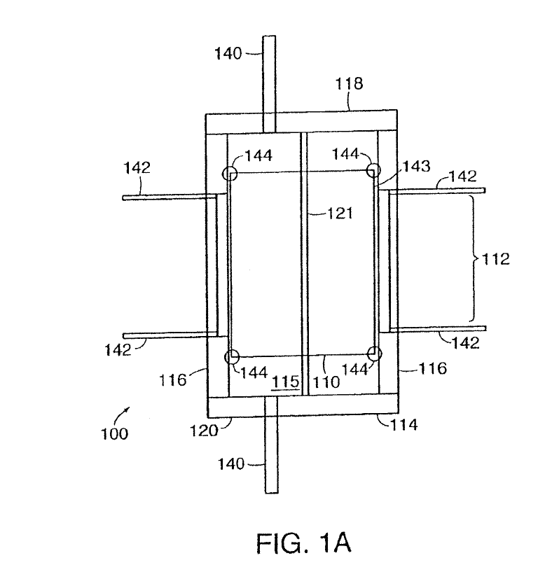

FIGS. 1 and 2 show front-sectional and top-sectional views, respectively, of a collider chamber apparatus 100 constructed according to the invention. FIG. 3 shows a perspective view of a portion of apparatus 100. Apparatus 100 includes a rotor 110 and a stator 112. The stator 112 is formed from part of a housing 114 (shown in FIG. 1) that encloses rotor 110. Housing 114 includes a cylindrical sidewall 116, a circular top 118, and a circular bottom 120. Top 118 and bottom 120 are fixed to sidewall 116 thereby forming a chamber 115 within housing 114 that encloses rotor 110. Rotor 110 is disposed for rotation about a central shaft 121 that is mounted within housing 114. Stator 112 is formed in a portion of sidewall 116.

As shown in FIG. 2, the cross section of stator 112 has a generally annular shape and includes an outer wall 122 and an inner wall 124. Outer wall 122 is circular. Inner wall 124 is generally circular, however, inner wall 124 defines a plurality of tear-drop shaped collider chambers 130. Each collider chamber 130 includes a leading edge 132, a trailing edge 134, and a curved section of the inner wall 124 connecting the leading and trailing edges 132, 134. For convenience of illustration, FIG. 3 shows only one of the collider chambers 130 in perspective. Further, FIG. 3 does not show the portion of housing 114 that extends above stator 112 and also does not show the portion of housing 114 that extends below stator 112.

The outer diameter of rotor 110 is often selected so that it is only slightly smaller (e.g., by approximately 1/5000 of an inch) than the inner diameter of stator 112. This selection of diameters minimizes the radial distance between rotor 110 and the leading edges 132 of the collider chambers 130 and of course also minimizes the radial distance between rotor 110 and the trailing edges 134 of the collider chambers 130.

Apparatus 100 also includes fluid inlets 140 and fluid outlets 142 for allowing fluid to flow into and out of the collider chambers 130. Apparatus 100 can also include annular fluid seals 144 (shown in FIG. 1) disposed between the top and bottom of rotor 110 and the inner wall of sidewall 116. Inlet 140, outlet 142, and seals 144 cooperate to define a sealed fluid chamber 143 between rotor 110 and stator 112. More specifically, fluid chamber 143 includes the space between the outer wall of rotor 110 and the inner wall 124 (including the collider chambers 130) of stator 112. Seals 144 provide (1) for creating a fluid lubricating cushion between rotor 110 and sidewall 116, (2) for restricting fluid from expanding out of chamber 143, and (3) for providing a restrictive orifice for selectively controlling pressure and fluid flow inside fluid chamber 143. The space in chamber 115 between bottom 114 and rotor 110 (as well as the space between top 118 and rotor 110) serves as an expansion chamber and provides space for a reserve supply of fluid lubricant for seals 144.

FIG. 1A shows an alternative embodiment of apparatus 100 in which fluid inlets 140 provide fluid communication between the environment external to apparatus 100 and chamber 115 through top 118 and bottom 120, and in which fluid outlets 142 a permit fluid communication between the environment external to apparatus 100 and the sealed chamber 143 through sidewall 116. Fluid inlets 140 may be used to selectively introduce fluid into chamber 115 through the top 118 and bottom 120, and some of the fluid introduced through inlets 140 may flow into sealed chamber 143. Fluid outlets 142 are used to selectively remove fluid from the sealed chamber 143. As those skilled in the art will appreciate, the fluid inlets and outlets permit fluid to flow into and out of, respectively, chamber 143 and may be arranged in many different configurations.

To simplify the explanation of the operation of apparatus 100, a simplified mode of operation will initially be discussed. In this simplified mode, fluid inlets and outlets 140, 142 are initially used to fill fluid chamber 143 with a fluid (e.g., water). Once chamber 143 has been filed, inlets 140 and outlets 142 are sealed to prevent fluid from entering or exiting the chamber 143. After fluid chamber 143 has been filed with fluid and sealed, a motor or some other form of mechanical or electrical device (not shown) drives rotor 110 to rotate about shaft 121 in a counter-clockwise direction as indicated by arrow 150 (in FIGS. 2 and 3). Rotation of rotor 110 generates local cyclonic fluid flow patterns in each of the collider chambers 130.

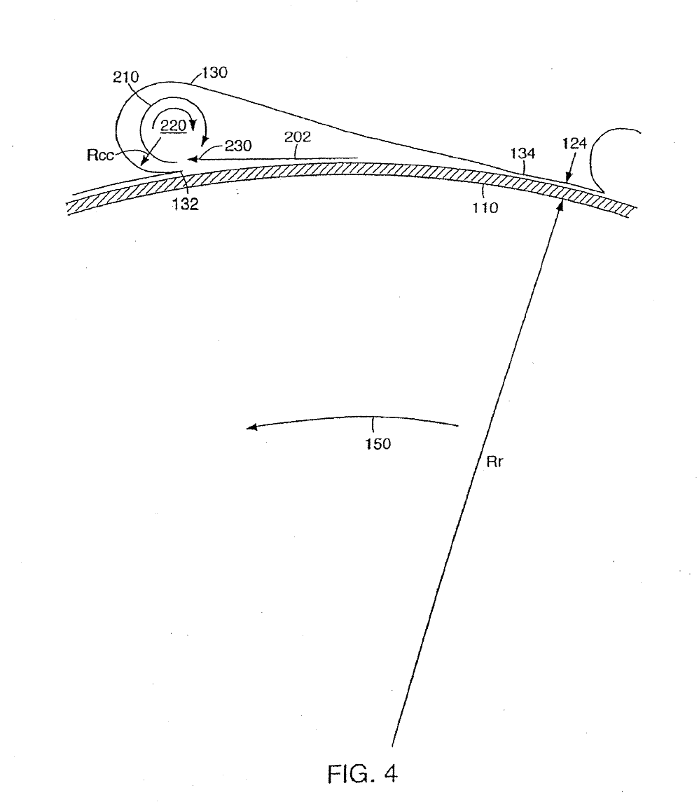

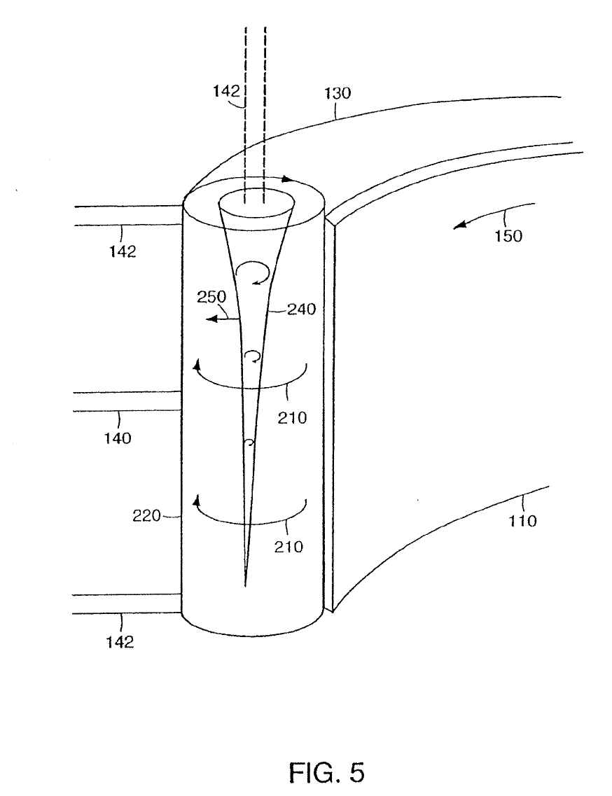

FIG. 4 shows a simplified top-sectional view of a portion of the fluid flow pattern in a single collider chamber 130 of apparatus 100. The rotation of rotor 110 in the direction of arrow 150 causes the fluid within chamber 143 to flow generally in the direction of arrow 150. Arrow 202 represents the trajectory of fluid molecules that are tangentially spun off of rotor 110 into collider chamber 130. These molecules are redirected by the wall of chamber 130 to flow in the direction of arrow 210 and form a cyclonic fluid flow pattern 220. Molecules flowing in pattern 220 flow generally in a clockwise direction as indicated by arrow 210. The rotational velocity of flow pattern 220 is determined by the rotational velocity of rotor 110, the radius of rotor 110, and the radius of the portion of chamber 130 within which pattern 220 flows. More specifically, the rotational velocity (e.g., in revolutions per minute) of flow pattern 220 is determined approximately according to the following Equation (1):

V_{α}∝(Rᵣ/R_{α})Vᵣ (1)

where V_{α} is the rotational velocity of pattern 220, Vᵣ is the rotational velocity of rotor 110, R_{α} is the radius of the portion of collider chamber 130 within which pattern 220 flows as indicated in FIG. 4, and Rᵣ is the radius of rotor 110. The radius R_{α} of collider chamber 130 is typically much smaller than the radius Rᵣ of rotor 110. Therefore, the rotational velocity V₄ of flow pattern 220 is normally much greater than the rotational velocity Vᵣ of rotor 110. In other words, apparatus 100 employs mechanical advantage, created by the disparity in the radii of rotor 110 and collider chamber 130, to greatly increase the rotational velocity of fluid flowing in chamber 130. In addition, the center of the roughly circular portion of collider chamber 130 can be located such that a circle formed by the outline of collider chamber would intersect a portion of rotor 110. Thus, in some embodiments, the widest portion of collider chamber is in the form of a “flattened” circle.

In one embodiment the radius Rᵣ of rotor 110 is six inches, the radius R_{α} of the portion of collider chamber 130 within which pattern 220 flows is one eighth (⅛) of an inch, the rotational velocity of the rotor is 3,400 revolutions per minute (RPM), and the rotational velocity of flow pattern 220 is approximately 163,200 RPM. Those skilled in the art will appreciate that 163,200 RPM is an enormous rotational velocity and is far higher than has been generated with prior art systems for manipulating fluid. For example, some centrifuges generate rotational velocities as high as 70,000 RPM, however, centrifuges do not approach the rotational velocities, and large centrifugal and centripetal forces, provided by the invention. Further, centrifuges provide only a single chamber for separation purposes whereas collider chamber apparatus 100 provides a plurality of collider chambers 130, all of which can accommodate a separately controllable cyclonic fluid flow for manipulating the fluid properties. Still further, centrifuges rapidly move a container of fluid but they do not move the fluid within the container relative to that container. Therefore, centrifuges do not greatly increase the number of molecular collisions occurring in the fluid contained within the centrifuge. In contrast to a centrifuge, an apparatus constructed according to the invention generates fluid flows that rotate at extremely high velocity relative to their containing collider chambers and as will be discussed in greater detail below thereby dramatically increases the number of molecular collisions occurring within the fluid contained within the apparatus.

The rotational velocity V_{α} discussed above is a macro-scale property of the cyclonic flow pattern 220. The velocities of individual molecules flowing in pattern 220 as well as the frequency of molecular collisions occurring in pattern 220 (i.e., the number of molecular collisions occurring every second) are important micro-scale properties of pattern 220. As is well known, the average velocity of molecules in a fluid (even a “static” or non-flowing fluid) is relatively high and is a function of the temperature of the fluid (e.g., 1500 feet per second for water at room temperature in a static condition). Typically, fluid molecules travel very short distances (at this high velocity) before colliding with other rapidly moving molecules in the fluid (e.g., the mean free path for an ideal gas at atmospheric pressure is 10⁻⁵ cm). The average molecular velocity and the average frequency of molecular collisions are micro-scale properties associated with any fluid. As will be discussed in greater detail below, operation of apparatus 100 dramatically increases the frequency of molecular collisions occurring in pattern 220 and also increases the velocities of molecules flowing in pattern 220, and thereby increases the temperature of fluid flowing in pattern 220.

Molecules flowing in pattern 220 continually collide with molecules that are spun into chamber 130 by rotor 110. In FIG. 4, the reference character 230 indicates the region where the maximum number of molecular collisions occurs between molecules flowing in pattern 220 and molecules that are spun off of rotor 110. The number of collisions added to the fluid in chamber 130 by operation of the invention is roughly proportional to the rotational velocity of the flow pattern 220 (i.e., since each molecule is likely to experience a new collision every time it traverses the circumference of the flow pattern and again passes through the location indicated by reference character 230). Therefore, the extremely high rotational velocity of cyclonic flow pattern 220 produces a correspondingly large number of molecular collisions. Such a large number of molecular collisions could not occur within a fluid in a static condition, and also could not occur within a fluid that does not move relative to its container (as in the case of a centrifuge).

A small amount of heat is generated every time a molecule flowing in pattern 220 collides with the wall of the collider chamber or with a molecule spun off of rotor 110. This heat results from converting kinetic energy of molecules flowing in pattern 220 into thermal energy. This energy conversion results in reducing the kinetic energy (or velocity) of molecules flowing in pattern 220, and if not for action of the rotor 110 the pattern 220 would eventually stop rotating or return to a static condition. However, rotor 110 continually adds kinetic energy to flow pattern 220 and thereby maintains the rotational velocity of pattern 220. The rotor 110 may be thought of as continually “pumping” kinetic energy into the molecules flowing in pattern 220, and the enhanced molecular collisions occurring in pattern 220 may be thought of as continually converting this kinetic energy into heat. As the apparatus 100 operates, the continuous generation of heat tends to increase the average molecular velocity of molecules flowing in pattern 220, and this increase in velocity further increases the number of molecular collisions occurring in pattern 220.

In the prior art, heat has been added to fluids and the molecular motion of the fluids have been increased in response to the added heat. In contrast to the prior art, the invention induces rapid motion in a fluid (i.e., the high macro-scale rotational velocity V_{α} of fluid in the collider chamber 130) and thereby generates heat in response to the increased motion. The invention therefore provides a fundamentally new way of heating, or adding energy to, fluids.

In a static fluid, molecular collisions are random in nature. In the collider chamber apparatus, the induced collisions are directional in nature. For example, as shown in FIG. 4, rotor 110 initially causes the fluid in chamber 143 to rotate in the direction indicated by arrow 150. Subsequently, some of the fluid is redirected by chamber 130 to flow in pattern 220. Since the fluid flow generated by rotor 110 in the direction of arrow 150 tangentially intersects the flow pattern 220, collisions between molecules flowing in pattern 220 and molecules spun off of rotor 110 consistently occur at the intersection of these two patterns indicated by reference character 230. Further, at the time of collision, the colliding molecules flowing in pattern 220 and spun off of rotor 110 are both moving in the same direction as indicated by arrow 202. This consistent occurrence, and the directional alignment of, molecular collisions within pattern 220 permit rotor 110 to continuously pump energy into flow pattern 220

Since flow pattern 220 is restricted to flow within collider chamber 130, the constant addition of heat to flow pattern 220 continuously increases both the pressure and the density of the fluid flowing in pattern 220. In summary, the combined effect of the unusually high macro-scale rotational velocity of pattern 220, the continuous addition of kinetic energy by rotor 110, and the confined space of the collider chamber 130 within which the pattern 220 flows is to greatly (1) increase the number of molecular collisions occurring in the fluid, (2) increase the temperature of the fluid, (3) increase the pressure of the fluid, and (4) increase the density of the fluid.

As stated above, operation of apparatus 100 dramatically increases the number of molecular collisions occurring in the fluid flowing in pattern 220. It is difficult to calculate the actual number of molecular collisions added by operation of the apparatus, however, this number of collisions may be estimated for an exemplary embodiment as follows. Assuming that a collider chamber is 6″ tall and that the molecules of fluid in the chamber have a height of 1/1000″, then approximately 6000 layers of fluid molecules are disposed in the collider chamber at any given instant. If the flow pattern within the collider chamber is rotating at 163,000 RPM, or 26,000 revolutions per second, then the chamber adds at least 156,000,000 (26,000×6000) molecular collisions every second, since each molecule on the periphery of the collider chamber will collide with a molecule spun off of rotor 110 every time the molecule completes a rotation around the collider chamber. A typical collider chamber apparatus an may include approximately 30 collider chambers, so operation of the apparatus adds at least 4,680,000,000 molecular collisions every second. It is understood that more or less molecular collisions may be obtained by varying the dimensions of the collider chamber and/or the speed or rotation of the rotor.

FIG. 5 shows a simplified perspective view of cyclonic fluid flow pattern 220 flowing in a collider chamber 130 that is provided with a central inlet 140, an upper outlet 142, and a lower outlet 142. Molecules flowing in pattern 220 rotate at a high rotational velocity in a clockwise direction as indicated by arrows 210. The high velocity, and the high number of collisions, of molecules flowing in pattern 220 rapidly heats the fluid in pattern 220. Some of the heated fluid vaporizes and the vaporized fluid tends to collect in a generally conical, or “cyclone shaped”, vapor region 240 towards the center of pattern 220. The vapor tends to collect near the center of pattern 220 because the large centrifugal force acting on mass flowing (or rotating) in pattern 220 tends to carry heavier (e.g., liquid) particles towards the perimeter of pattern 220 and correspondingly tends to concentrate lighter (e.g., gaseous or vapor) particles towards the center of pattern 220 where the centrifugal forces are reduced. The extremely high rotation velocity V_{α} of flow pattern 220 generates correspondingly large centrifugal forces at the periphery of pattern 220 and effectively concentrates the vapor in vapor region 240. Vapor region 240 tends to be conically shaped because the heated vapor tends to rise towards the top of chamber 230 thereby to expand the diameter of region 240 near the top of region 240.

As the vapor in region 240 increases in temperature (due to the increased molecular collisions occurring in pattern 220), the vapor tends to expand and thereby generates a force that acts radially in the direction indicated by arrow 250 on the liquid in pattern 220. This radial force tends to expand the outer diameter of flow pattern 220. However, the walls of collider chamber 130 (and the fluid molecules that are continuously spun off of rotor 110 to impact with pattern 220) provide external forces that prevent the outer diameter of pattern 220 from expanding. The net result of (1) the external forces that prevent the outer diameter of pattern 220 from expanding and (2) the radial force generated by the expanding vapor in vapor region 240 is to increase the pressure in flow pattern 220. The increased pressure tends to (1) compress the fluid flowing in pattern 220 to its maximum density, (2) increase the number of molecular collisions occurring in pattern 220, and (3) increase the heating of the fluid flowing in pattern 220.

In operation of apparatus 100, several factors tend to have a cumulative, combinatorial effect. For example, the continuous addition of kinetic energy by rotor 100 results in continuous generation of heat within apparatus 100. This continuous generation of heat tends to continuously increase the average velocity of molecules flowing within flow pattern 220. This continuous increase in molecular velocity tends to further increase the frequency of molecular collisions occurring within pattern 220 and thereby also leads to increased heat generation within apparatus 100. Still further, the increased heat tends to increase the pressure and density of the fluid flowing within pattern 220 and this increased pressure and density also tends to increase the number of molecular collisions occurring within pattern 220 and thereby also leads to increased heat generation. All of these factors combined are believed to provide for exponentially fast heating of fluid flowing within pattern 220.

One application of apparatus 100 is as a heater of fluids. Fluid delivered to collider chamber 130 by inlet 140 is rapidly heated. The heated fluid may be removed by outlet 142 and delivered for example to a radiator or heat exchanger (not shown) for heating either a building or applying heat to a process. The fluid exiting the radiator or heat exchanger may then of course be returned to inlet 140 for reheating in apparatus 100.

When used as a heater of fluids, it has been discovered that the operating efficiency of a metallic embodiment of apparatus 100, coupled to a metallic heat exchanger, increases over time with use of the same fluid in apparatus 100. That is, the amount of heat energy produced by apparatus 100 has increased with continued operation of apparatus 100 without a proportionate increase in the amount of electrical energy consumed to rotate rotor 110. Without being limited by any particular theory of operation, it is thought that operation of apparatus 100 induces chemical changes in the fluid in collider chamber 130. These chemical changes are theorized to promote the absorption of metallic species into the fluid from the metallic components of apparatus 100 and the metallic heat exchanger. As now described in greater detail, the addition of metallic species to the fluid is believed to increase the operating efficiency of apparatus 100.

As described above, heat is generated when the molecules of the fluid collide with each other or with surfaces of the rotor and/or stator, and at least a portion of the kinetic energy of the molecule is converted into thermal energy. Likewise, any particles that are in motion in the fluid also impart thermal energy when those particles collide with other particles or surfaces of the rotor and/or stator. The amount of energy produced is proportionate to the velocity of the molecule or particles as well as its mass.

Thus, increasing either or both of the velocity of the particles of the fluid or the mass of the particles in the fluid increases the amount of heat energy produced. When used as a heater of fluids, it is, therefore, advantageous to increase the mass of the particles of the fluid.

The metallic embodiment and heat exchanger described above were used as a test system for generating heat. Rotor 110 and stator 112 of apparatus 100 of the test system were cylindrical, as shown in FIG. 1. Apparatus 100 of the test system had 50 collider chambers 130. In the test system, fluid was delivered to collider chamber 130, heated, and removed from the collider chamber. The heated fluid was passed through a heat exchanger (not shown) and returned to collider chamber 130 to be reheated. Thus, the test system was a closed loop system with respect to the fluid. In the implementation of this particular test system, rotor 110 and stator 112 were constructed of aluminum. Thus, in this embodiment, the walls of collider chamber 130 were aluminum. Also, in this particular implementation, the heat exchanger that receives the heated fluid had metallic surfaces (e.g., tubing and heat exchange plates) containing copper and iron in contact with the fluid.

As stated above, it is believed that operation of the described test system caused metallic species to be absorbed into the collider fluid. The metallic apparatus 100 and metallic heat exchanger system described above was filled with water and operated on the order of hundreds of hours over a period of one year or more. In general, operation of the test system included a warm-up period and a steady state operation period. The warm-up period typically included circulating fluid through apparatus 100 and the heat exchanger at a flow rate of about 1.5 gallons per minute (GPM) and rotating rotor 110 at approximate 2500 RPM until the temperature of the fluid reached approximately 220° F. After reaching 220° F., the system would be operated in a steady state mode. During steady state operation, the rotor was rotated at about 1800 RPM and fluid was circulated through apparatus 100 and the heat exchanger at a flow rate of about 2 GPM.

Although the distilled water was substantially free of metallic species and had a slightly acidic pH before being subjected to collisions induced by operation of apparatus 100, a change in pH and the presence of metallic species was detected after operation of apparatus 100 of the test system. Table 1 shows results for three different fluid samples taken from the system after the operational period described above. Approximately one gallon of fluid total was removed from apparatus 100 for the three samples. Fluid sample 1 was taken from the system after the period of operation described above. Analysis of the sample shows increased pH as well as the presence of an elevated level of metallic species relative to the distilled water initially used in the system. Fluid sample 2 was taken during operation of the system. During the operational period, the test system was operated in the steady state condition described above. Analysis of sample 2 shows an increase in pH and metallic species relative to sample 1. Fluid sample 3 was taken from the system after the operational period during which fluid sample 2 was taken. Analysis of sample 3 shows that the metallic species present in that sample are generally equal to those present in the sample before the brief period of operation during which sample 2 was taken.

TABLE 1
Composition Analysis of Fluid Taken From Apparatus

| | Fluid Sample 1 | | Fluid Sample 2 | | Fluid Sample 3 | |
|---|---|---|---|---|---|---|
| Aluminum | 220 | mg/L | 310 | mg/L | 220 | mg/L |
| Iron | 3.9 | mg/L | 5.5 | mg/L | 4.1 | mg/L |
| Copper | 24 | mg/L | 35 | mg/L | 26 | mg/L |

| | | | |
|---|---|---|---|
| pH | 7.75 | 7.42 | 7.41 |
| Temperature | 75Deg. F. | 182Deg. F. | 100Deg. F. |

Because approximately one gallon of fluid was removed from apparatus 100 of the test system, an equal amount of water was added to apparatus 100 to return the test system to a full capacity. Thus, the concentration of metallic species (and any other particulates) in the fluid was reduced by approximately one-half. Apparatus 100 of the test system was then operated generally as described above for approximately one-half the amount of time that preceded the fluid exchange over a period of about six months.

Table 2 shows the results of analyses performed on fluid samples taken from apparatus 100 of the test system after the fluid exchange and operational period described above. As before, approximately one gallon of fluid total was removed from apparatus 100 for the three samples. Fluid sample 4 was taken from the system after the additional six months of operation described above. Analysis of the sample shows a pH nearly equal to that of that last fluid sample taken from the first test run (i.e., fluid sample 3). However, with the exception of iron content, the metallic species content was nearly half of that found in fluid sample 3.

Fluid sample 5 was taken during operation of the system. During the operational period, the test system was operated in the steady state condition described above. Analysis of sample 5 shows an increase in metallic species, total suspended solids, and density relative to fluid sample 4. Fluid sample 6 was taken from the system after the operational period during which fluid sample 5 was taken. An analysis of the metallic species and total suspended solids was not performed on fluid sample 6. However, it is observed that the pH and density of fluid sample 6 are increased from that found in fluid sample 5.

TABLE 2
Composition Analysis of Fluid Taken From Apparatus

| | Fluid Sample 4 | | Fluid Sample 5 | | Fluid Sample 6 |
|---|---|---|---|---|---|
| Aluminum | 100 | mg/L | 150 | mg/L | Not Tested |
| Iron | 3.6 | mg/L | 5.2 | mg/L | Not Tested |
| Copper | 12 | mg/L | 17 | mg/L | Not Tested |
| pH | 7.42 | | 7.04 | | 7.33 |
| Temperature | 71Deg. F. | | 180Deg. F. | | 100Deg. F. |
| Density | 1.06 | g/mL | 1.02 | mg/L | 1.07 mg/L |
| Total Suspended | 370 | mg/L | 620 | mg/L | Not Tested |
| Solids | | | | | |

Table 3 shows the results of analyses performed on the raw fluid (water) provided as makeup fluid to apparatus 100 of the test system before the second test run described above. As the analysis results of fluid sample 7 show, the level of metallic species present in the water is quite low compared to those found in the fluid within apparatus 100 of the test system after operation. Thus, it is concluded that the water is not a significant source of metallic species.

TABLE 3
Composition Analysis of Raw Fluid Makeup to Apparatus

| | Fluid Sample 7 | |
|---|---|---|
| Aluminum | 0.070 | mg/L |
| Iron | 0.038 | mg/L |
| Copper | 0.099 | mg/L |
| pH | 5.94 | |
| Temperature | 72Deg. F. | |
| Density | 1.00 | g/mL |

| | |
|---|---|
| Total Suspended | Not Tested |
| Solids | |

The analyses for the Aluminum, Iron, and Copper were performed according to EPA Method 200.7. The pH was determined according to EPA Method 150.1. The density was determined according to method SM 2710F. Total suspended solids were determined according to EPA Method 160.2.

Again, without being limited to any particular theory, it is thought that the collisions experienced by water molecules of the fluid in apparatus 100 causes some of the atoms of the water molecules to disassociate. This disassociation is thought to produce hydrogen free radicals, hydroxonium ions, and/or peroxides. Furthermore, the alkaline pH readings of the six fluid samples taken from the test system are believed to indicate the possible formation of metal hydroxides. It is further contemplated that the formation of hydrogen peroxide in the fluid of apparatus 100 can lead to the creation of metal oxides through a reaction between the hydrogen peroxide and metallic components of the system.

It is noted that Aluminum, Copper, and Iron are considered to be insoluble in hot and cold water. Thus, the presence of these metallic species in the fluid after prolonged operation of apparatus 100 further supports the theories set forth above. Moreover, the elevated amount of Aluminum in the fluid relative to the amounts of Copper and Iron are thought to be attributable to the fact that the energy of the fluid molecules is highest in collider chambers 130, which are constructed of Aluminum in the test system. Furthermore, by maintaining the fluid in a closed system, the metallic species and particles accumulate, thereby increasing the benefits.

In addition to the chemical changes thought to take place due to operation of apparatus 100 on the fluid therein, it is theorized that metallic colloids are formed and suspended in the fluid. That is, microscopic and non-ionic metallic particles become suspended in the fluid in apparatus 100.

As both ionic and colloidal metallic species are carried by the fluid during operation of apparatus 100, these metallic species experience a high rate of collisions due to the extremely high rotational velocity of the fluid within which the species are suspended. However, because the mass of the metallic species are greater than the mass of the water molecules alone, each collision of a metallic species imparts more energy, and thus, more heat, into the fluid. Thus, it is the creation of these relatively higher molecular weight particles (as compared to water alone) that is thought to be responsible for the increase in operating efficiency over time. Furthermore, it is believed that further operation of apparatus 100 on the fluid contained therein increases the metallic species content of the fluid, thereby further increasing the efficiency of operation.

In addition to increasing the density of the fluid by causing ionic and colloidal species to enter the fluid, the density of a fluid exhibiting any amount of compressibility can be increased by maintaining the fluid under an increased pressure. Thus, by increasing the density of the fluid entering a collider chamber, the total amount of mass entering the collider chamber is increased. Therefore, as described above, the total number of molecular collisions increase, thereby generating more heat than in a fluid of lower relatively density. If apparatus 100 is included in a closed system, the fluid can be maintained under pressure by pressurizing the entire system. In some embodiments, pressure variations throughout the system are minimized. It is theorized that this contributes to maintaining desirable characteristics in the fluid that contribute to the total energy imparted into the fluid by apparatus 100. However, the fluid entering apparatus 100 can also be maintained under pressure by providing a backpressure device (e.g., a valve) on the outlet of the collider chambers of apparatus 100 and pumping the fluid into the inlet of the collider chambers under pressure.

The pressure of the fluid circulating through the test system can be maintained at several atmospheres or higher (i.e. about 14.696 pounds per square inch absolute (PSIA) or higher). When circulating a liquid through apparatus 100, this has the added advantage of reducing the amount of liquid that boils due to the increase in the boiling point of the liquid due to the increase in the pressure of the fluid. By reducing the amount of liquid that becomes vapor in the collider chambers, the amount of mass in the collider chambers is increased relative to what would be expected at lower relative pressures.

Another example of a use for apparatus 100 is as a separator. For example, apparatus 100 may be used to separate water from a contaminated waste stream. As an example, fluid waste delivered via inlet 140 is heated inside collider chamber 130. Heated water vapor tends to rise to the top of chamber 130 whereas the solid waste portion contained in the fluid tends to separate and drop to the bottom. The concentrated and separated heavier waste product may be removed via the lower outlet 142 and the heated water vapor may be removed via the upper outlet 142. For such an application it may be desirable to provide a fluid outlet 142 of the type shown in dashed lines in FIG. 5 that permits withdrawal of vapor from the top of vapor region 240. This removed heated water vapor is sufficiently hot to be flash evaporated under a vacuum condition at a relatively low ambient temperature (e.g., at room temperature). The evaporated water vapor may then be condensed and filtered into pure water to complete the separation process. This same process may be applied to desalinization of sea water to separate the salt and other mineral compounds to produce a potable water for human consumption or other uses. Alternatively, instead of evaporating water and leaving the waste behind, if the waste product (e.g., alcohol) vaporizes at a lower temperature than water, the waste can be evaporated and separated from the water first and condensed in the same manner. In such a case, a water-alcohol waste stream could be continuously introduced into the collider apparatus via inlet 140, purified water could be continuously removed from the lower outlet 142 and alcohol vapor could be continuously removed from the upper outlet 142.

As another example of a useful separation process, apparatus 100 may be used to separate mercury from a waste water stream. Wastewater containing mercury compounds are a serious health concern and the technology for consistently removing mercury to below detectable levels of 2 ppb is currently underdeveloped. As is known, mercury in a wastewater stream may be placed into an ionic state by addition of chemicals (e.g., chlorine) to the wastewater stream. Apparatus 100 can be used to heat such a wastewater stream to a temperature above the evaporation point of mercuric chloride and below the evaporation point of the water fraction of the wastewater. The mercury, in the form of mercuric chloride, may then be removed from apparatus 100 by evaporation and may then be condensed and filtered prior to final fluid disposal.

As yet another example of a useful separation process, apparatus 100 may be used to remove reclaimable salts from process wastewater. For example, metallic salts used in the plating industry may be removed from wastewater by using apparatus 100 to flash evaporate the water as generally described above. Such removal of these salts permits recovered dean water (i.e., the water evaporated by operation of apparatus 100 and subsequently condensed and if desired filtered) to be reused in the process rather than being discharged into a sewer and also permits the reclamation and reuse of the salts. Since such a process dramatically reduces the amount of waste disposed, into a sewer or otherwise, apparatus 100 offers significant benefits in pollution control.

In still another useful separation process, apparatus 100 may be used in the production of precious metals (e.g., gold, silver, platinum, iridium). Although not commonly known, conventional refining techniques sometimes only extract about 10% of the precious metal content from the concentrated precious metal bearing ores and, consequently, waste slags produced during the mining and smelting of concentrated precious metal bearing ores sometimes contain over 90% of the original precious metal content of the ore. These precious metals are still chemically bonded to, as an example, the iron sulfide mineral structure contained in the waste slag material. As described below, apparatus 100 may be used to extract more of the precious metal from the waste slag.

In one process, the waste slag is initially reduced to a fine powder. A heated solution of water and sulfuric acid is then circulated through the powder to release the iron/precious metal sulfides. The solution can be continually leached through the slag powder to form a leachate containing metallic sulfides dissolved into solution with the water-sulfuric acid mixture. The leachate is then treated within apparatus 100. As discussed generally above, operation of apparatus 100 will heat the leachate within the apparatus. Gaseous oxygen and if desired an appropriate catalyst is then added to the heated leachate within apparatus 100 to permit the oxygen to react with the dissolved metallic sulfides and thereby produce sulfur trioxide (SO₃). This reaction also converts the metallic sulfides into metallic oxides and water. The sulfur trioxide may then be removed from apparatus 100. After removal of the sulfur trioxide, the material remaining within apparatus 100 is primarily water and metallic oxides. The water may be flash evaporated as discussed generally above to permit extraction of the metallic oxides. The metallic oxides may then be processed using conventional chemical or metallurgical techniques to extract the precious metals from the oxides. The sulfur trioxide removed from the apparatus 100 may also be added to water to form sulfuric acid (H₂ SO₄), which can of course be used for preparing more leachate. As those skilled in the art will appreciate, apparatus 100 provides a convenient and efficient mechanism for converting the metallic sulfides to metallic oxides as discussed above.

Another example of a use for apparatus 100 is as a chemical reaction accelerator. The increased molecular collisions occurring within flow pattern 220 will increase the rate of reaction of any reactants flowing within pattern 220. To further increase reaction rates, it may be desirable to coat the outer wall of rotor 110, or the inner wall 124 of stator 112 with an appropriate catalyst or reagent.

As yet another example, apparatus 100 may be used to disassociate molecular bonds and thereby facilitate a chemical reaction occurring within the apparatus. More specifically, the increased high energy molecular collisions occurring within apparatus 100 may be used to disassociate molecular bonds and thereby to chemically alter the fluid contained within apparatus 100. If desired, this process may be enhanced by addition of selected chemical catalysts or reagents. As an example, if a mixture of alcohol, water, and an aluminum oxide catalyst is input to apparatus 100, the increased molecular collisions caused by operation of apparatus 100 can separate water from alcohol and form ethylene. So as shown by this example, apparatus 100 may be used to chemically alter a compound introduced into apparatus 100. In this example, since the evaporation point of ethylene is lower than the evaporation point of water, following the catalytic disassociation of water and alcohol, apparatus 100 may be used to flash evaporate the ethylene as described generally above and to thereby physically change the alcohol into ethylene. So generally, apparatus 100 may be used to chemically separate, or change, a compound into two or more distinct and different chemical compounds, and may then be subsequently used to physically separate those compounds from each other.

Chemical reactions can be classified as being either exothermic or endothermic depending upon whether Gibbs free-energy change (AG) is negative or positive. Endothermic reactions require energy input in order to convert reactants (substrates) into one or more products. Consider the following reaction: A+B+energy→C. This reaction is an example of an endothermic reaction where “A” and “B” are reactants and require energy input in order to overcome the energy of activation to form product “C”. Activation energy is that amount of energy necessary to reach the transition state. The transition state comprises an activated complex, i.e., the reactants transforming into product. The transition state is the highest level of energy for a given reaction. Often a practitioner will employ a catalyst which effectively serves to lower the energy of activation. Examples of catalysts are metals and enzymes. Catalysts are often used up in a particular reaction. (However, biological catalysts, enzymes, often survive the reaction.)

In the present invention, apparatus 100 can be used to generate sufficient energy to over come the energy of activation and drive the reaction to the right, i.e., the formation of product. One attractive feature is that the energy generated by apparatus 100, e.g., heat energy, can be used for as long as the apparatus is operational. Without undue experimentation, a practitioner can ascertain the appropriate parameters for apparatus 100 that are necessary to drive a particular reaction, and as long as the apparatus is operational, the particular reaction can run indefinitely. Under the present invention, there is no need to replace a particular catalyst. Moreover, due to the ability to separate molecular species using the present apparatus 100, one may be able to separate product from reactants.

One example of applying this invention is in the preparation of chemical compounds used in the pharmaceutical industry. As discussed herein, a suitable media (water, saline, and the like) employed to manufacture a particular compound can first be de-contaminated and/or purified using the apparatus described herein. Then the reactants can be added under conditions suitable to generate a compound. The apparatus herein described is capable of producing sufficient energy to effectively drive the reaction from reactants to product. This process can continue indefinitely so long as the apparatus 100 is operational. Energy generated by apparatus 100 and not used to facilitate the reaction can be applied to other purposes.

The present apparatus 100 not only provides sufficient energy to drive a reaction, but it also increases the incidence of molecular collision. Such collision can occur with sufficient energy as to favor the formation of product. One skilled in the art will appreciate the importance in any given chemical reaction of increasing the incidence of molecular collision. Additionally, having these molecular collisions occurring with sufficiency of energy as to favor product formation is advantageous.

One skilled in the art will appreciate that this method can be applicable on the nano-scale dimension. Without undue experimentation, a practitioner can determine suitable parameters for operating apparatus 100 in an appropriate manner to facilitate reactions at this level.

In a related application, this invention is directed toward a method of mixing fluids or molecular compounds with a fluid(s). As described herein, the present invention can be used for separation processes, however, under suitable conditions, it can be used for mixing. The apparatus 100 can be used to facilitate the mixing of two or more solutions. There may be no apparent thermodynamic barrier to the mixing of these solutions, however, solutions comprising water and oil do have thermodynamic considerations. Under suitable conditions, solutions with challenging thermodynamic features can be admixed having different degrees of homogeneity. Moreover, molecular compounds (chemical compounds, e.g., pharmaceutical agents) can be admixed with other compounds or individually with a particular medium(s). Examples of suitable mediums include, but are not limited to, water, oil, saline, organic solutions, etc.

Obviously, industries other than the pharmaceutical industry can benefit from this invention such as the cosmetic industry, nano-material industry, chemical industry, paint industry (e.g., apparatus 100 can facilitate the mixing of paint having multiple components), and the like.

An example of such a use for collider apparatus 100 is to treat hazardous fluids such as PCB's or fluids containing other hazardous compounds such as dioxins. In such cases, the increased molecular collisions, heat, pressure, and density produced by apparatus 100, in addition to selected addition of chemical reagents or catalysts, may be used to disassociate molecular bonds in the fluid and to thereby separate the compound input to apparatus 100 into two or more chemically distinct compounds. Following this chemical separation, apparatus 100 may subsequently be used to flash evaporate one or more of the chemical compounds and thereby to physically separate the constituent compounds.

Fluids used in biomedical research or medical therapy can often be contaminated with one or more microorganisms. Such fluids include, but are not limited to, water, cell and tissue culture media, plasma, pharmaceutical carriers, and the like. The present apparatus 100 can be used to inactivate or kill microorganisms. Microorganisms such as bacteria and viruses are well known to be susceptible to heat inactivation. (See, Biology of Microorganisms (2000) Prentice Hall (9th ed.), pp. 742-745, the entire teaching of which is incorporated herein by reference.) For example, it was shown that Legionella pneumophila can be heat inactivated at around 60° C. (See, Muraca et al., Applied and Environ. Micro., (1987) v 53, no. 2, pp. 447-453, the entire teaching of which is included herein by reference.) Bacteria, even with their cell wall component, can be heat inactivated. Temperatures around 50° C. to about 70° C. can be used to inactivate many bacterial species. However, temperatures equal to or exceeding 100° C. are used to inactive/kill bacterial pathogens that are resistant to lesser temperature treatment. Often these higher temperatures are necessary in order to kill spores. Another parameter to be considered is the time of exposure to elevated temperature. Often one may employ a lower temperature for an extended period time to inactivate or kill certain bacterial species. Temperature and time parameters for various infectious agents are well understood by those skilled in the art.

Sterilization is not always the goal. Historically, pasteurization has been very effective in destroying all non-spore forming infective agents in heat-sensitive foods such as milk, other dairy products and liquid egg products. Pasteurization typically involves a lesser heat treatment which better maintains product quality by killing only part of the microbial population present in a food source, e.g., milk. Food products are often subjected to pasteurization rather than sterilization. Apparatus 100 can effect the pasteurization of food products. Food products can be subjected to apparatus 100 under conditions suitable for pasteurization. These conditions are well known to those skilled in the art. One cautionary note, even with food products it may be necessary to inactivate completely infectious agents that are classified as only pathogens, such as hepatitis A virus. Contamination of food products such as milk, cream etc. by hepatitis A is of concern. This virus is susceptible to heat inactivation and can be attenuated in various food products by treating them with the present invention. (See, e.g., Bidawid, et al., J. Food Prot. (2000) 63(4), pp. 522-8, the entire teaching of which is incorporated in its entirety by reference herein.)

Viruses are also susceptible to heat inactivation. It is well known by those skilled in the art that the pathogenicity of viruses can be attenuated by elevated temperatures. The viruses used for vaccine preparations are often heat inactivated. Viral particles exposed to, e.g., 50° C. and above can often be inactivated. (See, e.g., Harper, et al. J. Virol., 26(3), pp 646-659, the entire teaching of which is incorporated herein by reference.) Viruses such as HIV can be heat inactivated. (See, e.g., Einarsson, et al, Transfusion (1989) 29(2), pp. 148-152, the entire teaching of which is incorporated herein by reference.) Obviously this has significant clinical application for non-cell bearing fluids used in the clinical setting, e.g., plasma, intravenous fluids, and the like. A significant concern in the clinical setting is the administration of fluids that may be contaminated with potentially deadly pathogens, both bacterial and viral.

As can be appreciated from the above discussion, apparatus 100 can be used to facilitate the elimination and/or inactivation of both bacteria and viruses. However, parasitic organism can also be subjected to inactivation using elevated temperatures. A concern in many situations is fluid contamination. Subjecting this fluid to the apparatus 100 under conditions suitable to inactivate or kill microorganisms can be effected by facilitating elevated temperatures within the apparatus 100. As stated above, the apparatus generates internal temperatures that can meet or exceed those required to inactivate or kill microorganisms. And under the appropriate conditions, temperature and time, microorganisms can be eliminated from a particular fluid. In essence, apparatus 100 can be used to facilitate sterilization.

Apparatus 100 can be employed to treat fluids prior to their introduction into a subject, whether those fluids are therapeutic in nature or food items. The present invention can be used to sterilize, or pasteurize, fluids prior to their introduction into a subject (including human) or use in, e.g., biomedical research. Fluids contaminated with pathogens can cause serious aliments if not death both at the cellular level as well as the organism level. By first subjecting these fluids to the present invention, these pathogenic agents can be attenuated or completely inactivated.

It will be appreciated by those skilled in the art that the inactivation of microorganisms may not only be facilitated by the heat generated by apparatus 100, but also by the shear stress induced by the apparatus.

Examples of other suitable applications include, but are not limited to, realizing a high degree of pathogenic safety in structures such as buildings that utilize, e.g., circulating water for maintaining environmental conditions. This circulating water can be subjected to apparatus 100 under suitable conditions to inactive/kill pathogens such as Legionella, as well as other pathogens. A corollary to employing apparatus 100 in this manner is that the energy generated (e.g., in form of heat) can be converted into other forms of energy or used as a heat source.

Apparatus 100 can be housed in various settings. It can be in, e.g., a hospital, a hotel, a research facility, a food manufacturing plant, a commercial structure (e.g., office building), a residential home, etc. Also, it can be housed on an ocean going vessel (including a ship or submarine), airplane, terrestrial vehicle, planetary space vehicle, and the like. This apparatus can be used to decontaminate and/or purify fluids while at the same time generate energy that can be used for other purposes, e.g., serve as a heat source.

The present invention can be used to augment or assist heating systems used to control environmental conditions in a public, commercial, industrial, or residential facility, not to mention ocean going vessels and passenger vehicles. Apparatus 100 can be employed to preheat condensate return water used in a facility's boiler feed-water system. This could significantly reduce the steam load demand and the associated cost. Further, by using apparatus 100 in the process, the environmental pollution burden is lessened by reducing the emission of greenhouse gases. Apparatus 100 can be used to reclaim waste heat from a facility's waste steam condensate which can be routed through apparatus 100. Not only can the return water be re-heated, it can also undergo de-contamination and purification, as described above. Apparatus 100 can be disposed in-line along a facility's environmental control system (e.g., heating system).

As described above, it can be advantageous to maintain the fluid circulating through apparatus 100 at a pressure higher than ambient. When put to use in a boiler system, the water passing through apparatus 100 can be maintained at 5 pounds per square inch gauge (PSIG) for feed into pre-boiler holding tank. In addition, apparatus 100 can be used to reheat condensate return, maintained at 30 PSIG. Maintaining the liquid under pressure increases the mass of fluid in the collider chambers of apparatus 100 as well as reducing the flashing of the liquid water into steam in various parts of the boiler system. The pressures listed above are provided for illustration only, as embodiments of apparatus 100 are capable of operating at pressures above and below those disclosed, for example, at or above hundreds of PSIG or below atmospheric.

As those skilled in the art will appreciate, in addition to the simple methods of operation described above, apparatus 100 may be operated according to many different methods. For example, instead of rotating the rotor 110 at constant rotational velocity, it may be desirable to vary the rotor's rotational velocity. In particular, it may be advantageous to vary the rotor's rotational velocity with a frequency that matches a natural resonant frequency associated with the fluid flowing in flow pattern 220. Varying the rotor's rotational velocity in this fashion causes the frequency of molecular collisions occurring in pattern 220 to oscillate at this natural resonant frequency. Altering the frequency of molecular collisions in this fashion permits optimum energy transfer to the fluid flowing in pattern 220. Molecular collisions occurring at the fluid's natural resonant frequency facilitates weakening and disassociation of molecular bonds between molecules in the fluid allowing for the withdrawal of selected molecular compounds from the fluid mass flowing in pattern 220 as was discussed above.

As another example of variations from the basic embodiments of apparatus 100, rather than using a cylindrical rotor, it may be advantageous to use a rotor having a non-constant radius (e.g., a conically shaped rotor). Using a rotor with a non-constant radius induces different velocities and different frequencies of molecular collisions in different portions of the chamber 130.

As yet another example of variations in apparatus 100, the fluids used in apparatus 100 may be pressurized by pumping or other means prior to introduction into chamber 143. Using pressurized fluids in this fashion increases the density of fluid in pattern 220 and increases the frequency of molecular collisions occurring in pattern 220. Alternatively, fluids may be suctioned into apparatus 100 by the vacuum created by the centrifugal forces within apparatus 100. As still another example, fluids may be preheated prior to introduction to apparatus 100. When apparatus 100 is used as part of a system, it may be advantageous to use heat generated by other parts of the system to preheat the fluid input to the apparatus. For example, if apparatus 100 is used to vaporize water and thereby separate water from a waste stream, heat generated by a condenser used to condense the vaporized water may be used to preheat the fluid input to apparatus 100.

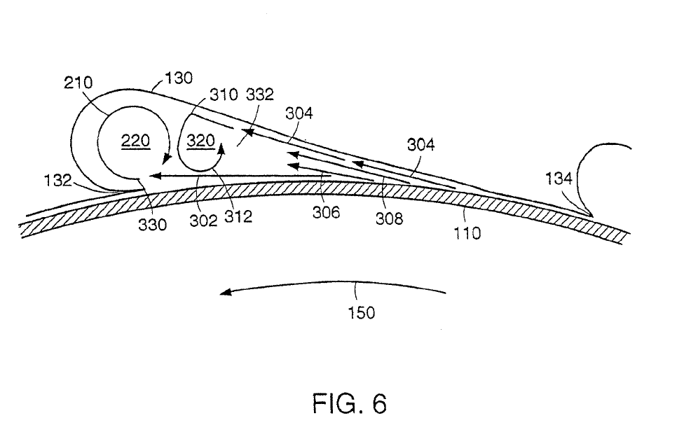

FIG. 6 is similar to FIG. 4, however, FIG. 6 shows a more detailed top view of the fluid flow pattern in a single collider chamber 130. Arrows 302, 304, 306, 308 illustrate the trajectory of fluid molecules that are spun tangentially off of rotor 110 into collider chamber 130. Arrow 302 shows the trajectory of molecules that are thrown into collider chamber proximal leading edge 132. These molecules tend to collide with and enter cyclonic fluid flow pattern 220. Arrow 304 shows the trajectory of fluid molecules that are spun off of rotor 110 into chamber 130 proximal the trailing edge 134. These molecules tend to impact cyclonic fluid flow pattern 220 as indicated at reference character 310. Impact with flow pattern 220 tends to redirect these molecules in the direction indicated by arrow 312 and these molecules tend to form a secondary cyclonic flow pattern 320. Arrows 306 and 308 show the trajectory of fluid molecules that are spun off of rotor 110 into the center of collider chamber 130. These molecules tend to collide with the secondary cyclonic flow pattern 320.

There are several regions of enhanced molecular collisions in the flow patterns illustrated in FIG. 6. One such region is indicated by reference character 310. This region is where molecules in secondary cyclonic flow pattern 320 impact molecules flowing in the primary cyclonic flow pattern 220. Reference character 330 indicates another region of enhanced collision. This region is where molecules flowing in primary cyclonic flow pattern 220 tend to collide with molecules that are spun off of rotor 110. Finally, reference character 332 indicates another region of enhanced collision. This region is where molecules flowing in secondary cyclonic flow pattern 320 tend to collide with molecules spun off of rotor 10. The enhanced molecular collisions in all of these multiple cyclonic regions contribute to the increased heating of the fluid in collider chamber 130.

The properties of secondary cyclonic flow pattern 320 are similar to those of primary cyclonic flow pattern 220. The fluid flowing in the primary and secondary cyclonic flow patterns 220,320 becomes heated and pressurized. However, since the radius of secondary cyclonic flow pattern 320 tends to be smaller than the radius of primary cyclonic flow pattern 220, the fluid flowing in pattern 320 tends (1) to rotate faster, (2) to experience more molecular collisions, and (3) to become heated more quickly, than the fluid flowing in pattern 220.

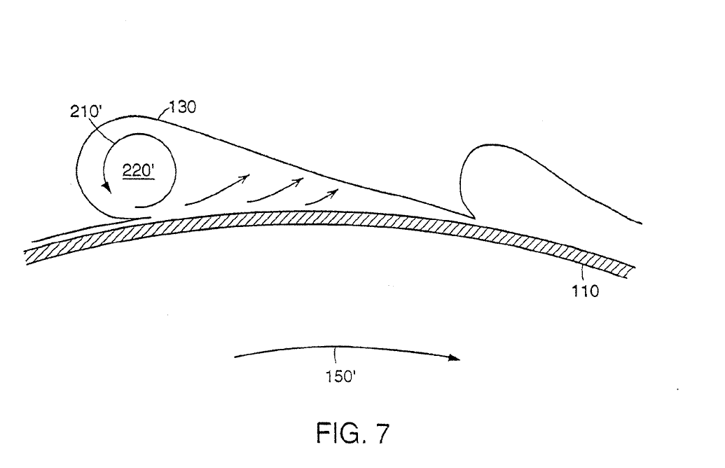

As is shown in FIG. 6, when tear-drop shaped collider chambers are used, it is desirable to rotate rotor 110 in a direction that is towards the leading edge 132. However, as is shown in FIG. 7, the invention will still operate in such a configuration even if rotor 110 is rotated in the opposite direction. As shown in FIG. 7, opposite rotation of rotor 110 will still generate at least one cyclonic flow pattern 220′ collider chamber 130.

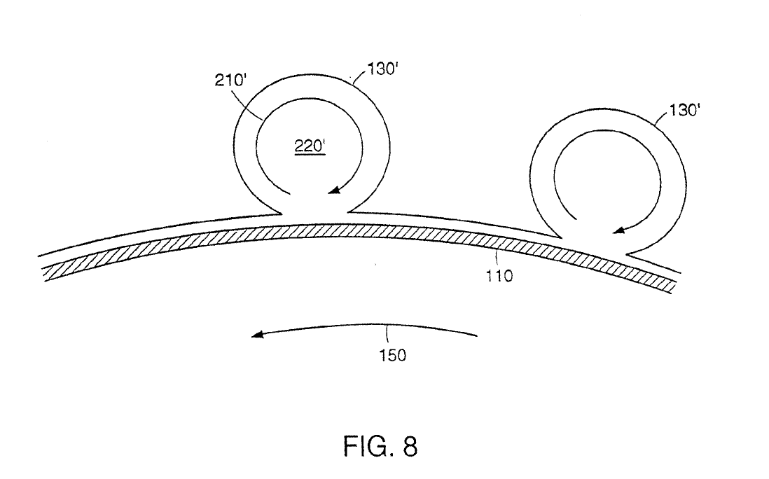

The tear-drop shape (as shown in FIG. 6) is one shape for the collider chambers 130. However, as shown in FIG. 8, other shaped collider chambers may be used. For example, FIG. 8 shows a top-sectional view of a C-shaped (or circular) collider chamber 130′. Rotation of rotor 110 will generate a single cyclonic flow pattern 220′ each such shaped collider chamber 130′.

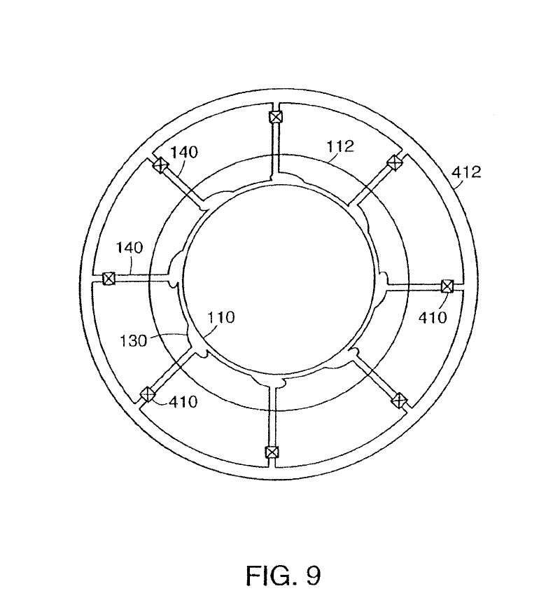

FIG. 9 shows a sectional-top view of one configuration of the apparatus 100 constructed according to the invention. In this configuration, each collider chamber 130 is provided with a corresponding fluid inlet 140 for introducing fluid into the collider chamber. Each fluid inlet is fluidically coupled to a manifold 412. Each fluid inlet is also provided with a valve 410 for selectively controlling the fluid flow between its respective collider chamber 130 and the manifold 412. Each collider chamber 130 can also be provided with a fluid outlet (not shown) and each of the fluid outlets can be provided with a valve for selectively controlling the amount of fluid leaving the chamber 130. Providing each collider chamber 130 with its own fluid inlet, fluid outlet, and control valves allows the conditions (e.g., temperature or pressure) in each of the collider chambers 130 to be independently controlled. However, each collider chamber 130 of apparatus 100 need not have separate inlet, outlet, and corresponding valves unique to each collider chamber 130. As explained in detail below, the inlet and outlet of more than one collider chamber may be joined.

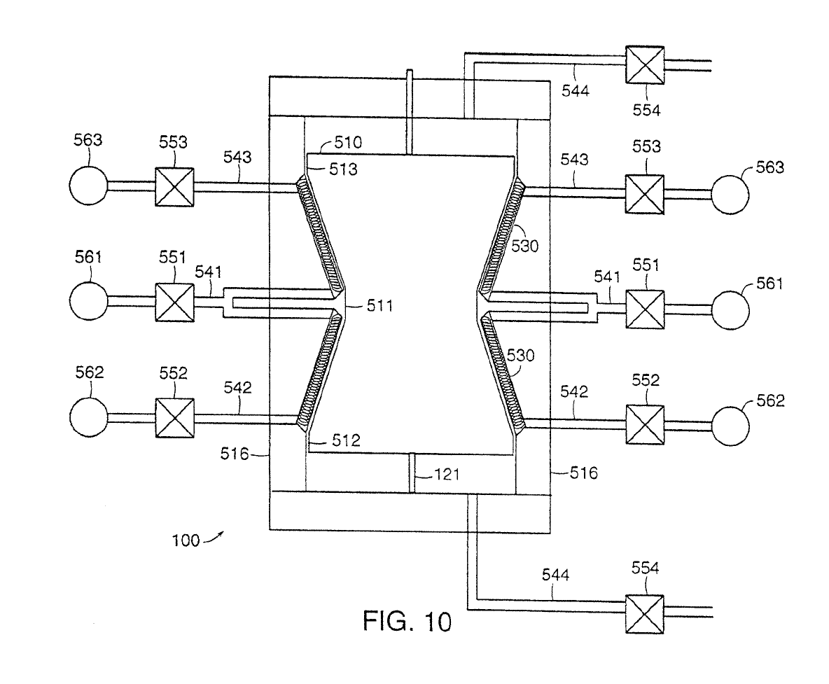

FIG. 10 shows a sectional-side view of another embodiment of a collider chamber apparatus 100 constructed according to the invention. In this embodiment, the apparatus includes an “hour-glass shaped” rotor 510 disposed for rotation about shaft 121. Rotor 510 includes a middle portion 511, a bottom portion 512, and a top portion 513. The outer diameter of the middle portion 511 is smaller than the outer diameter of the top and bottom portions 512, 513. The apparatus further includes a sidewall 516 that defines a plurality of collider chambers 530 extending vertically along the periphery of the rotor 510. The apparatus further includes inlets 541 that allow fluid to enter the collider chambers 530 near the middle portion 511 of the rotor 10. The apparatus also includes outlets 542 and 543 that allow fluid to exit from the collider chambers 530 near the bottom and top portions 512 and 513, respectively. In one embodiment, the apparatus is constructed as is illustrated generally in FIG. 9 with a plurality of collider chambers surrounding the outer periphery of the rotor and with each collider chamber 530 being provided with its own inlet 541 and its own outlets 542, 543. Each of the inlets 541 can be coupled to a manifold 561 via a control valve 551. Similarly, each of the outlets 542 and 543 can be coupled to manifolds 562 and 563, respectively, via control valves 552 and 553, respectively. Apparatus 100 may also include additional fluid inlets/outlets 544 for permitting fluid introduction and removal through the apparatus' top and bottom. These inlets/outlets 544 may also be provided with control valves 554.

In operation, the centrifugal force, and compression, generated by rotation of rotor 510 is greater near the top and bottom portions 513, 512 than near the middle portion 511. So, fluid provided to the collider chambers 530 via the inlets 541 is suctioned into the apparatus and is naturally carried by the centrifugal force generated by rotor 510 to the outlets 542, 543.

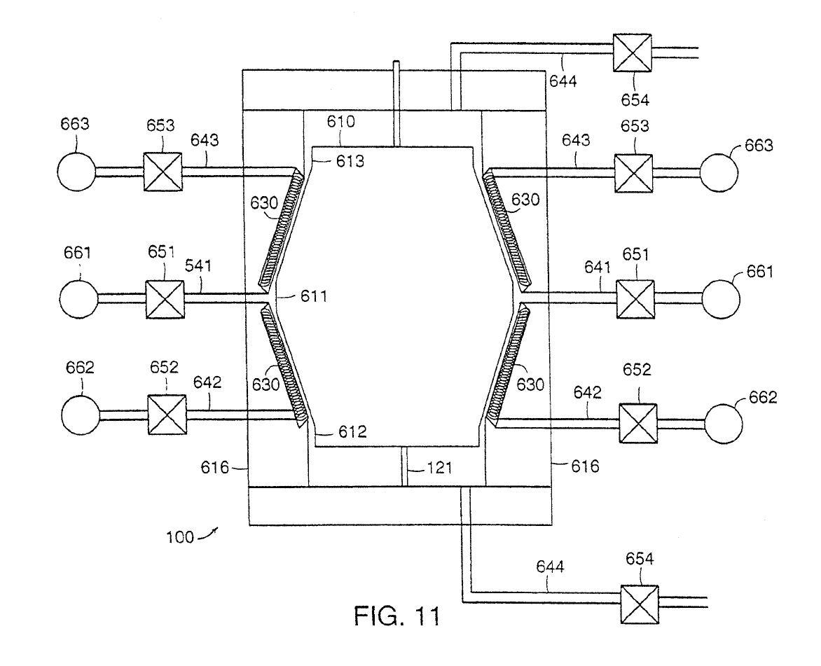

FIG. 11 shows a sectional side view of yet another embodiment of a collider chamber apparatus 100 constructed according to the invention. In this embodiment, the apparatus includes a rotor 610. Rotor 610 is generally cylindrical or barrel shaped, and rotor 610 includes a middle portion 611, a bottom portion 612 and a top portion 613. The outer diameter of middle portion 611 is greater than the diameters of bottom and top portions 612, 613. The apparatus further includes a sidewall 616 that defines a plurality of collider chambers 630 extending vertically along the periphery of the rotor 610. The apparatus further includes outlets 641 that allow fluid to exit the collider chambers 630 near the middle portion 611 of the rotor 610. The apparatus also includes inlets 642 and 643 that allow fluid to enter from the collider chambers 630 near the bottom and top portions 612 and 613, respectively. In one embodiment, the apparatus is constructed as is illustrated generally in FIG. 9 with a plurality of collider chambers surrounding the outer periphery of the rotor and with each collider chamber 630 being provided with its own outlet 641 and its own inlets 642, 643. Each of the outlets 641 can be coupled to a manifold 661 via a control valve 651. Similarly, each of the inlets 642 and 643 can be coupled to manifolds 662 and 663, respectively, via control valves 652 and 653, respectively. Apparatus 100 may also include fluid inlets/outlets 644 for permitting fluid introduction and removal through the apparatus' top and bottom. These inlets/outlets 644 may also be provided with control valves 654.

In operation, the centrifugal force generated by rotation of rotor 610 is greater near the middle portion 611 than near the top and bottom portions 613, 612. So, fluid provided to the collider chambers 630 via the inlets 642, 643 is naturally carried by the centrifugal force generated by rotor 610 to the outlets 641.

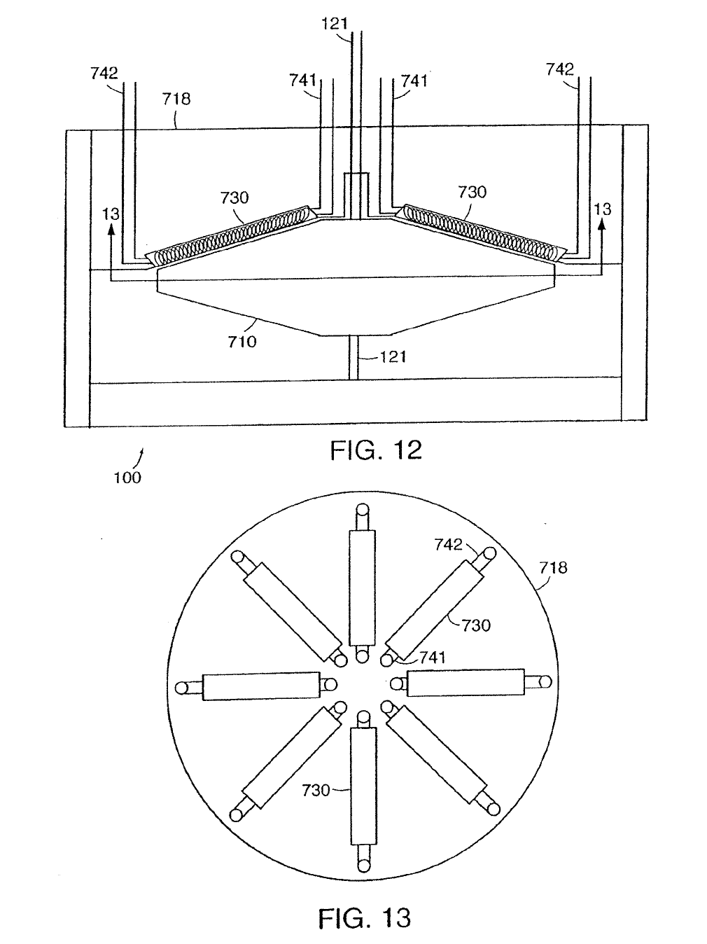

FIG. 12 shows a sectional-side view of yet another embodiment of a collider chamber apparatus 100 constructed according to the invention. This embodiment includes a generally disk shaped rotor 710 disposed for rotation about shaft 121 and a top 718 that defines a plurality of generally horizontal collider chambers 730 that extend along an upper surface of rotor 710. FIG. 13 shows a view of top 718 taken in the direction of line 13-13 shown in FIG. 12. Each of the collider chambers 730 is provided with an inlet 741 and an outlet 742. Centrifugal force generated by rotation of rotor 710 tends to carry fluid provided to collider chamber 730 via inlet 741 to the outlet 742. In some embodiments, each of the inlets and outlets is provided with its own control valve (not shown).

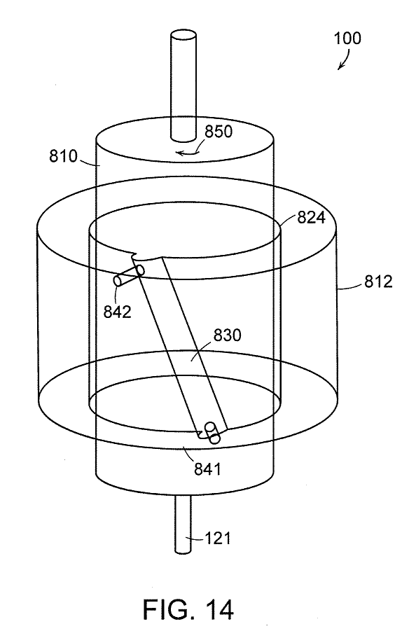

The collider chambers in the various embodiments of collider chamber apparatus 100 described above have a substantially linear axis about which the fluid inside the collider chamber rotates. However, in one implementation of the collider chamber apparatus 100, each collider chamber has an axis that is helical. FIG. 14 shows a perspective view of a collider chamber apparatus 100 with a collider chamber 830 that twists along an inner wall 824 of a stator 812. While only a single helical collider chamber 830 is shown for the sake of simplicity of the figure, it is understood that multiple helical collider chambers can be included in this implementation.

As in the embodiments described above, this illustrative implementation has a rotor 810 disposed for rotation about a shaft 121. The collider chamber 830 is provided with an inlet 841 and an outlet 842. Because the helical collider chamber 830 has a longer path between inlet 841 and outlet 842 than is possible with a linear collider chamber in an equally sized stator 812, the fluid residence time in the helical collider chamber 830 is greater than that in the linear collider chamber. Thus, it is believed a greater amount of energy can be imparted to the molecules of the fluid in the helical collider chamber 830, resulting in the generation of more heat as compared to that produced in a linear collider chamber.

FIG. 14 shows the outlet 842 as being located approximately 60 degrees apart from the inlet 841 in a direction of rotation 850. However, the inlet 841 and outlet 842 of helical collider chamber 830 can be separated by a greater or lesser angle and still be within the scope of the invention. For example, helical collider chamber 830 can pass along the entire circumference of the stator 812 such that the outlet 842 is located above the inlet 841. Moreover, helical collider chamber 830 may pass along the circumference of stator 812 in a clockwise or counterclockwise direction.

When helical collider chamber 830 passes along the circumference of stator 812 in the same direction as the rotation of rotor 810, the frictional force generated by rotation of rotor 810 not only causes rotation of the fluid within the collider chamber 830, but also tends to carry the fluid provided to collider chamber 830 via inlet 841 to the outlet 842. In some embodiments, each of the inlets and outlets is provided with its own control valve (not shown).

Although FIG. 14 shows a cylindrical stator 812 and rotor 810 combination, it is understood that the helical collider chamber implementation can be used in any of the embodiments of collider chamber apparatus 100 described above. For example, the hour-glass-shaped rotor 510 show in FIG. 10, the barrel-shaped rotor 610 shown in FIG. 11, and/or the disk-shaped rotor 710 shown in FIG. 12 can be implemented with helical collider chambers.

Those skilled in the art will appreciate that the collider chambers illustrated in FIGS. 10-14 may be used to generate cyclonic fluid flows of the type generally illustrated in and described in connection with FIG. 5. FIGS. 10-14 have been presented to illustrate a few of the numerous embodiments of collider chamber apparatuses that are embraced within the invention.

As discussed above, collider chamber apparatuses constructed according to the invention may be used for a variety of purposes. The collider chamber apparatus provides for a diverse treatment of fluids, including liquids, gasses, slurries, and mixtures thereof. Inducing motion in a fluid to increase the molecular collisions occurring in the fluid and to thereby produce fundamental changes in the fluid's properties (e.g., change of temperature or chemical structure) is accomplished by creating directional flows within the fluid. Molecular collisions in a static fluid can only be random in nature. Molecular collisions in the collider chamber apparatus are directional in nature resulting in enhanced controllability of the properties of the fluid not before achievable. The use of induced motion to control the frequency of molecular collisions and the ability to alter the state of the fluid in a uniform manner thus allows for precise control of the fluid's desired properties.

In different embodiments, the face of rotor 110 may be smooth, scoriated (i.e., scored with a cross-hatch pattern) or treated to increase capillary flow for the fluid. The rotor may also be treated to provide for catalytic reactions occurring within apparatus 100. Further, apparatus 100 may be constructed from a variety of materials including metallic, thermoplastic, mineral, fiberglass, epoxy, and other materials. It may be desirable to base the selection of the materials used to construct apparatus 100 on the fluids that will be used in the apparatus and/or the potential use to which apparatus 100 will be put.

For example, one embodiment of apparatus 100 is constructed of aluminum and thermoplastic. In this embodiment, stator 112 is constructed of polyvinylidene fluoride (commercially available as Kynar® from Arkema, Inc.), which is a thermoplastic. This particular thermoplastic is desirable because of its resistance to abrasion, its strength, and high thermal stability. However, thermoplastic embodiments are not limited to this material, and the use of other thermoplastics is within the scope of the invention. The thermoplastic stator 112 is relatively light in comparison to many metals and increases the transportability of apparatus 100. Additional benefits are realized when such an apparatus 100 is used to generate heat in a fluid. Namely, the thermoplastic has a relatively high insulation value and overall lower heat capacity. Thus, less of the heat generated in the fluid within collider chambers 130 escapes the fluid due to heat loss from the external surface of stator 112.

Rotor 110 described above is constructed of aluminum and is hollow. Both of these characteristics contribute to a reduction in weight of apparatus 100 and reduce the amount of mass of apparatus 100 that absorbs heat produced in the fluid in collider chambers 130. Thus this particular embodiment has a relatively short “warm-up” period during which rotor 110 and stator 112 absorb the heat produced before arriving at the temperature of the fluid (approximately one-half of the test system described above). In addition, because the rotating mass is reduced, the amount of energy required to spin rotor 110 is reduced, thereby improving the efficiency of apparatus 100.

It is expected that the metal and thermoplastic embodiment described above would cause similar effects to take place in the fluid circulated therein upon operation of apparatus 100. In addition, it is expected that the energy imparted in the molecules of the fluid would cause particles of the thermoplastic to enter the fluid. Due to the relatively higher molecular weight of the thermoplastic molecules (relative to the fluid alone), each collision of the thermoplastic molecules would impart high levels of energy into the fluid. Thus, it is expected that increases in efficiency would be realized with prolonged operation of the metal and thermoplastic apparatus 100.

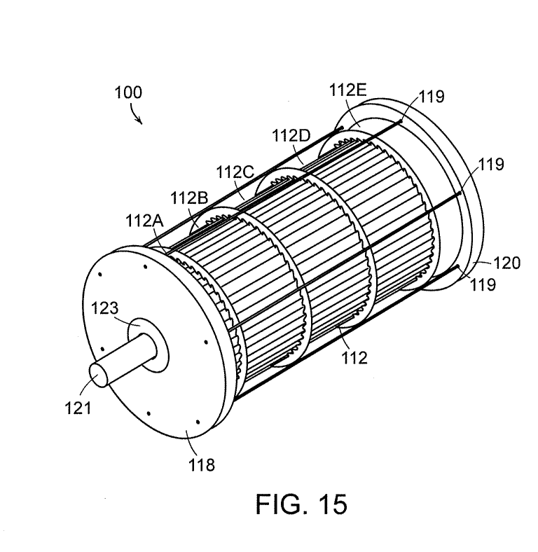

In the embodiments illustrated in FIGS. 1-3 and 9-14, the stators (e.g. 112 of FIGS. 1-3) are shown as monolithic. However, the stators need not be composed of a single piece. In some implementations, the stators can be constructed of several pieces that are held together. FIG. 15 is a perspective view of an embodiment of apparatus 100 with a stator 112 that is constructed of stator segments 112A-E. Stator segments 112A-E are shown in FIG. 15 as semi-transparent to illustrate the tear-drop shaped collider chambers defined by the inside walls of each segment. Stator segments 112A-E have a generally annular shape, and are held together by a clamping force imparted by circular top 118 and circular bottom 120. Clamping rods 119 pass between circular top 118 and circular bottom 120 and provide tension to draw top 118 and bottom 120 together. Clamping rods 119 can attach directly to each of top 118 and bottom 120 by a threaded connection, or clamping rods 119 may pass through holes in each of top 118 and bottom 120 and be secured thereto by threaded nuts (not shown).

FIG. 15 also illustrates central shaft 121 passing through top 118. Although not shown, central shaft 121 passes through bottom 120 as well. A fluid seal 123 is disposed on central shaft outside top 118. Likewise, although not shown, a fluid seal is also provided on the opposing end of central shaft 121 outside bottom 120. The fluid seals allow central shaft 121 to pass outside the cavity created by stator segments 112A-E, top 118, and bottom 120 while maintaining a sealed fluid cavity. The fluid seals may be configured to pass a small amount of fluid for cooling and wetting of the seals.

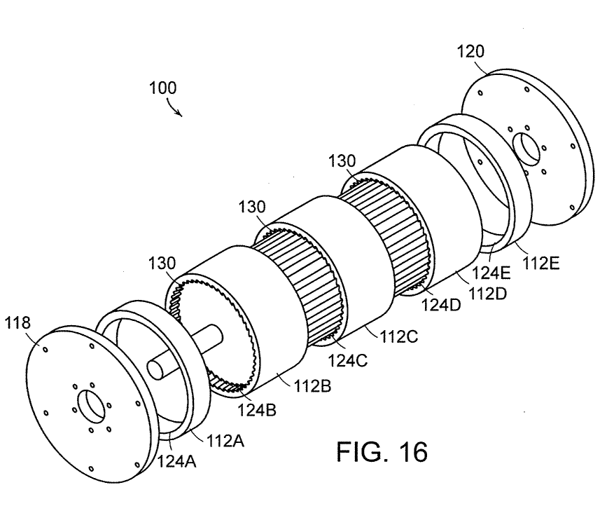

FIG. 16 is an exploded perspective view of the embodiment of apparatus 100 shown in FIG. 15. Seal 123 and clamping rods 119 are omitted for clarity. Each of stator segments 112A-E has a corresponding inner wall 124A-E. Inner walls 124A-E are generally circular and define a plurality of tear-drop shaped collider chambers 130. Inner walls 124B-D of segments 112B-D define tear-drop shaped chambers along the length of the segments, while segments 124A and 124E act as “caps” at opposing ends of those chambers. Thus, when segments 124A-E are held together (as shown in FIG. 15), annular seals similar to seals 144 of FIG. 1 are maintained at the top and bottom of each collider chamber 130.

Although not shown in the figures, it is understood that the outside geometry of the stator is not limited to a circular shape. For example, in some embodiments, the outside cross-section of the stator may be square, rectangular, or another shape. This is true of both the monolithic stator and segmented stator. Thus, stator segments 112A-E shown in FIGS. 15-16 could be formed from a square or rectangular plate of metal that has been machined to create the collider chambers described above. In such an embodiment, channels can be created in the corners of the plate through which may pass clamping rods 119.

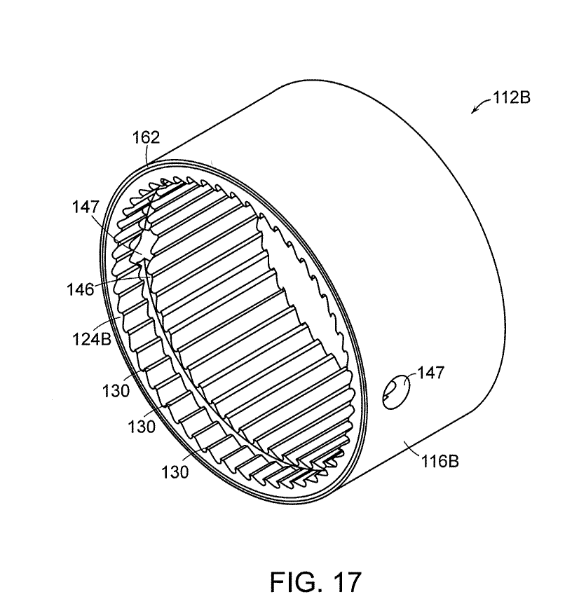

FIG. 17 is a perspective view of stator segment 112B. As described above, inner wall 124B of stator segment 112B defines a portion of collider chambers 130. Inner wall 124B of stator segment 112B also defines a inner raceway 146 that provides a fluid connection between collider chambers 130. Stator segment 124B also has a outlet port 147 that passes through a sidewall 116B and provides a fluid connection to inner raceway 146. Thus, outlet port 147 and inner raceway 146 cooperate to provide a fluid pathway from each of collider chambers 130 to the outside of apparatus 100, with inner raceway 146 serving as a fluid manifold for each of collider chambers 130. Although not shown, stator segment 112E can have a similar raceway and inlet port. Stator segment 112B also includes a lip 162 that aids in alignment between stator segment 112B and other segments. Lip 162 can also be lined with a gasket material to create a fluid seal.

Inlet and outlet piping and valves (not shown) can be attached to the inlet and outlet ports to control fluid flows into and out of collider chambers 130. The inner raceways and fluid ports can be used alone to supply fluid circulation to apparatus 100, or they can be used in combination with the other methods for introducing fluid into and removing fluid from collider chambers 130 described above. It is understood that inner raceway 146 and outlet port 147 may also be used in any of the other embodiments described herein and need not be limited to embodiments having a segmented stator 112.

Since certain changes may be made in the above apparatus without departing from the scope of the invention herein involved, it is intended that all matter contained in the above description or shown in the accompanying drawing shall be interpreted in an illustrative and not a limiting sense.

What is claimed is:

1. A method comprising:
disposing a fluid comprising a metals content of more than about 100 mg/L between a stator and a rotor, the stator including an inner wall, the inner wall defining a plurality of collider chambers, and the rotor including an outer wall that is proximal to the stator inner wall; and
rotating the rotor, relative to the stator, about an axis, rotation of the rotor in a first direction relative to the stator causing the fluid in each of the collider chambers to rotate within the collider chamber in a second direction opposite to the first direction, rotation of the rotor causing the temperature of the fluid in the collider chambers to increase.

2. The method of claim 1, the metallic species being ionic.

3. The method of claim 1, the metallic species being colloidal.

4. The method of claim 1, the metallic species being at least one of aluminum, copper, and iron.

5. The method of claim 1, the metallic species comprising more than about 350 mg/L of the fluid.

6. The method of claim 1, at least one of the rotor and stator comprising a metal and the rotating the rotor, relative to the stator, causing the metal of the at least one of the rotor and stator to enter the fluid.

7. The method of claim 1, further comprising providing the fluid comprising the metals content of more than about 100 mg/L.

8. The method of claim 1, the fluid further comprising a total suspended solids of more than about 370 mg/L.

9. The method of claim 8, the fluid further comprising a total suspended solids of more than about 619 mg/L.

10. A method comprising:
disposing a fluid comprising a total suspended solids of more than 370 mg/L between a stator and a rotor, the stator including an inner wall, the inner wall defining a plurality of collider chambers, and the rotor including an outer wall that is proximal to the stator inner wall; and
rotating the rotor, relative to the stator, about an axis, rotation of the rotor in a first direction relative to the stator causing the fluid in each of the collider chambers to rotate within the collider chamber in a second direction opposite to the first direction, rotation of the rotor causing the temperature of the fluid in the collider chambers to increase.

11. The method of claim 10, the suspended solids comprising more than about 619 mg/L of the fluid.

12. The method of claim 10, the rotating the rotor, relative to the stator, causing suspended solids to enter the fluid.

13. The method of claim 10, further comprising providing the fluid comprising total suspended solids of more than 370 mg/L.

14. The method of claim 10, the suspended solids comprising plastic particulates.

15. A method comprising:
disposing a fluid between a stator and a rotor, the stator including an inner wall, the inner wall defining a plurality of collider chambers, and the rotor including an outer wall that is proximal to the stator inner wall;
rotating the rotor, relative to the stator, about an axis above a predetermined rotational speed for a cumulative predetermined amount of time, the cumulative predetermined amount of time being at least about 24 hours; and
after rotating the rotor for the cumulative predetermined amount of time, rotating the rotor, relative to the stator, about the axis, rotation of the rotor in a first direction relative to the stator causing the fluid in each of the collider chambers to rotate within the collider chamber in a second direction opposite to the first direction, rotation of the rotor causing the temperature of the fluid in the collider chambers to increase.

16. The method of claim 15, the predetermined rotational speed being at least about 180° rotations per minute.

17. The method of claim 15, the cumulative predetermined amount of time being at least about 100 hours.

18. The method of claim 15, further comprising:
removing at least a portion of the fluid from at least one of the collider chambers; and
passing at least a portion of the fluid removed from the collider chambers through a heat exchanger system; the heat exchanger system and the stator being a closed system.

19. A method comprising:
providing a stator and a rotor, the stator including an inner wall, the inner wall defining a plurality of collider chambers, and the rotor including an outer wall that is proximal to the stator inner wall;
delivering a fluid into at least one of the collider chambers, the fluid comprising a metals content of more than about 100 mg/L;
rotating the rotor, relative to the stator, about an axis, rotation of the rotor in a first direction relative to the stator causing the fluid in each of the collider chambers to rotate within the collider chamber in a second direction opposite to the first direction; and
withdrawing the fluid from at least one of the collider chambers.

20. The method of claim 19, further comprising removing heat from the fluid withdrawn from the at least one of the collider chambers.

21. The method of claim 19, further comprising increasing a pressure of the fluid above a predetermined pressure before delivering the fluid to the at least one of the collider chambers, the predetermined pressure being about 14.7 pounds per square inch absolute.

22. The method of claim 21, the predetermined pressure being about 44.7 pounds per square inch absolute.

23. The method of claim 19, further comprising decreasing a pressure of the fluid below a predetermined pressure before delivering the fluid to the at least one of the collider chambers, the predetermined pressure being about 14.7 pounds per square inch absolute.

24. A method comprising:
providing a stator and a rotor, the stator including an inner wall, the inner wall defining a plurality of collider chambers, and the rotor including an outer wall that is proximal to the stator inner wall;
delivering a fluid into at least one of the collider chambers, the fluid comprising a total suspended solids of more than 370 mg/L;
rotating the rotor, relative to the stator, about an axis, rotation of the rotor in a first direction relative to the stator causing the fluid in each of the collider chambers to rotate within the collider chamber in a second direction opposite to the first direction; and
withdrawing the fluid from at least one of the collider chambers.

25. The method of claim 24, further comprising removing heat from the fluid withdrawn from the at least one of the collider chambers.

26. The method of claim 24, further comprising increasing a pressure of the fluid above a predetermined pressure before delivering the fluid to the at least one of the collider chambers, the predetermined pressure being about 14.7 pounds per square inch absolute.

27. The method of claim 26, the predetermined pressure being about 44.7 pounds per square inch absolute.

28. The method of claim 24, further comprising decreasing a pressure of the fluid below a predetermined pressure before delivering the fluid to the at least one of the collider chambers, the predetermined pressure being about 14.7 pounds per square inch absolute.

29. A method comprising:
providing a stator having an inner wall, the inner wall defining a plurality of collider chambers;
providing a rotor disposed for rotation about an axis, an outer wall of the rotor being proximal to the inner wall of said stator;
introducing a putatively contaminated fluid into a space between the inner wall of the stator and said outer wall of the rotor, the contaminated fluid comprising an infectious agent selected from the group consisting of bacteria, virus, parasite, and a combination thereof; and
rotating the rotor within the stator to generate a rotational flow of the fluid in each of the collider chambers, the rotational flow of the fluid in each of the collider chambers causing the temperature of at least portion of the fluid contained within each collider chamber to increase.

30. The method of claim 29, the fluid being selected from the group consisting of water, cell media, tissue media, plasma, and a pharmaceutical carrier.

31. The method of claim 29, the increase in temperature being sufficient for pasteurization of said fluid.

32. The method of claim 31, the fluid being a food source.

33. The method of claim 29, further comprising collecting the decontaminated fluid.
